(19) United States
(12) Patent Application Publication
(10) Pub. No.: US 2015/0196520 A1
(43) Pub. Date: Jul. 16, 2015

(54) METHOD AND PHARMACEUTICAL COMPOSITION FOR TREATING DERMATOPHYTOSIS

(71) Applicant: Shiro YOSHIZAKI, Tokushima (JP)

(72) Inventor: Shiro YOSHIZAKI, Tokushima (JP)

(21) Appl. No.: 14/663,646

(22) Filed: Mar. 20, 2015

Related U.S. Application Data
(63) Continuation-in-part of application No. PCT/JP2013/074503, filed on Sep. 11, 2013.

(30) Foreign Application Priority Data
Sep. 21, 2012 (JP) 2012-208794

(51) Int. Cl. A61K 31/198; A61K 9/00

(52) U.S. Cl. CPC A61K 31/198; A61K 9/0014

(57) ABSTRACT

The present invention provides a drug that is effective in treating and/or completely curing dermatophytosis with ease. More specifically, the present invention provides a dermatophytosis pharmaceutical composition characterized in that it is topically applied and includes, as a main active ingredient, at least one organic acid salt selected from the group consisting of ethylenediaminetetraacetic acid salts.

### TECHNICAL FIELD

The present invention relates to a pharmaceutical composition for dermatomycosis. More specifically, the present invention relates to a pharmaceutical composition for dermatomycosis containing, as an active ingredient, an organic acid salt that is capable of bonding strongly with magnesium ions and calcium ions.

### BACKGROUND ART

It is well known that some organic acids have an antimicrobial effect against microorganisms. For example benzoic acid and sorbic acid are widely used as preservatives in food products, cosmetics, pharmaceuticals, and the like. It is also known that benzoic acid and the like have a therapeutic effect against dermatophytosis (e.g., tinea pedis), a representative form of dermatomycosis. However, it is not well known that salts of organic acids have an antimicrobial effect against pathogenic fungi that are the cause of dermatomycosis (dermatophytes, Candida, Malassezia, and the like).

Organic acids used to treat dermatomycosis include benzoic acid, undecylenic acid (10-undecenoic acid), acetic acid, and citric acid, which are known as therapeutic agents for treatment of dermatophytosis. Because these organic acids have been used for some time, there is no patent literature stating that these organic acids alone have a therapeutic effect against dermatophytosis. However, there is some patent literature showing the therapeutic effects against tinea of mixtures of these organic acids with other antimicrobial active substances. Examples of such patent literature include literature disclosing a topical treatment for tinea consisting of miconazole nitrate or econazole nitrate to which benzoic acid or a salt thereof is added as an auxiliary (Patent Literature 1); literature disclosing an antifungal agent where undecylenic acid is combined with an imidazole antifungal agent (Patent Literature 2); and literature disclosing a mixture of bamboo vinegar solution with 1% citric acid (Patent Literature 3).

As therapeutic agents for dermatomycosis consisting primarily of salts of an organic acid, there are patents for therapeutic agents for dermatophytosis that include zinc salts, calcium salts, magnesium salts, or copper salts of undecylenic acid. However, such patents do not describe the action mechanism by which these salts act on the dermatophytes, or how these salts are related to the undecylenic acid that is the basis of the antimicrobial action (Patent Literature 4 and 5).

The following seven documents that relate to dermatophytosis can be given as examples of patent documents describing the relationship between organic acid salts and dermatomycosis. Although a topical treatment for tinea consisting of miconazole nitrate or econazole nitrate to which benzoic acid or a salt thereof is added as an auxiliary has been disclosed, there is no specific description of the benzoate (Patent Literature 1). A formulation of terbinafine and diclofenac or indomethacin has been disclosed, with undecylenic acid and a salt thereof included as an antimicrobial agent; however, there is no specific description of the undecylenic acid and the salt (Patent Literature 6). “Qiora Inner Serum R (dispenser type),” an eau de cologne massage gel product from Shiseido Co., Ltd., cures the tinea of sufferers, and a mixture of all eleven of the components of this product has been disclosed as a therapeutic agent for tinea. Although sodium citrate and edetate are included in such components, it is not known which of the eleven components have a curative effect on tinea, and this cannot be ascertained. Note that although sodium citrate as mentioned in this patent is the name in common usage, the correct name is citric acid trisodium. It is believed that the edetate is a typically used edetate disodium salt (Patent Literature 7). A combination of the well-known tinea remedy lanoconazole and zinc undecylenate has also been disclosed (Patent Literature 8). A nail patch that includes sodium acetate as an agent to facilitate penetration of the well-known athlete's foot remedy terbinafine into the nail has been disclosed (Patent Literature 9). It has been disclosed that the antimicrobial effect of the well-known tinea remedy bifonazole is improved by adding an auxiliary (sodium citrate, bismuth gallate, bismuth subnitrate). However, it is unclear why the antimycotic activity of bifonazole is improved with such auxiliaries, and the role of the citric acid trisodium as an auxiliary agent has also not been established (Patent Literature 10). Although an antimycotic agent that is a mixture of guaiazulene, which has bactericidal effects, and sorbic acid or a salt (sodium salt, potassium salt, calcium salt) thereof has been disclosed, the embodiments describe only one kind of drug that combines guaiazulene and sorbic acid, and there is no description of the sorbic acid salts (Patent Literature 11). In the patents mentioned above, the organic acid salts that are used are all added as auxiliary agents to well-known antifungal remedies; or the inclusion of an organic acid salt is merely mentioned, and no substantial description thereof is provided.

On the other hand, one patent document that discusses the relationship between organic acid salts and mycoses other than dermatophytosis discloses that edetic acid salts such as disodium calcium edetate act as a therapeutic agent for vaginal candidiasis (Patent Literature 12). It has also been disclosed that edetic acid salts such as tetrasodium edetate suppress the formation of a biofilm (pellicle) of Candida or the like (Patent Literature 13 and 14). Although such patent literature discloses that edetic acid salts suppress Candida, there is no mention of cutaneous candidiasis. No patent literature that discusses the relationship between organic acid salts and malasseziosis could be found.

A significant factor showing that organic acids have antimicrobial effects is the decrease in pH derived from the acidity of the organic acid. Further, it is believed that damage results from the decrease in pH inside microbes due to non-ionized, non-disassociated organic acid being taken inside the microbes, or due to antimicrobial effects peculiar to organic acids (Non-patent Literature 1 and 2). Organic acids that are used in dermatomycosis therapeutic agents include benzoic acid and undecylenic acid. Benzoic acid is combined with salicylic acid in Whitfield's Ointment, which is used in antifungal therapy for dermatophytosis (British Medical Association, 1998, Non-patent Literature 3). In China, an ointment having a similar compound is used under the name Hua Tuo Cream; however, benzoic acid is not registered as an antifungal agent in the Japanese Pharmacopoeia. Undecylenic acid, with 11 carbon atoms, is an organic acid that has obtained approval in Japan as a therapeutic agent for dermatophytosis; however, it is rare in clinical use (Non-patent Literature 4). Acetic acid (vinegar, pyroligneous acid, bamboo vinegar) is used as a traditional medicine in the treatment of dermatophytosis (tinea). Citric acid is also known for its use as a folk remedy (Non-patent Literature 5); however, there is also a scientific paper indicating that citric acid exhibits no antimicrobial effect against fungus (Non-patent Literature 6). As described above, since the antimicrobial effect of organic acids is thought to derive from non-disassociated organic acid molecules, it is understood that the salts of organic acids have no antimicrobial effect. For example, regarding benzoic acid, in Concise International Chemical Assessment Document No. 26, “Benzoic Acid and Sodium Benzoate,” the Executive Summary states that “[t]he two compounds are being considered together because it is undissociated benzoic acid that is responsible for its exhibits antimicrobial activity. As benzoic acid itself is only slightly soluble in water, sodium benzoate—which, under acidic conditions, converts to undissociated benzoic acid—is often used instead” (Non-patent Literature 7). Although ethylenediaminetetraacetic acid (edetic acid) has weak antimicrobial effects against gram-negative bacteria and fungi, it cannot be used alone as a preservative, and is used in combination with other preservatives (Non-patent Literature 8). It is understood that organic acid anions, which result from ion disassociation of organic acid salts, do not penetrate the cell walls of microbes. Therefore, it is generally believed that the salts of organic acids do not have an antimicrobial action.

Recently, it has been reported that edetate disodium has an antimicrobial effect on Candida (Non-patent Literature 9) and Aspergillus (Non-patent Literature 10). Further, it has been reported that various kinds of edetate formulations have an antimicrobial effect against Candida (Non-patent Literature 11).

### CITATION LIST

### Patent Literature

PTL 1: Japanese patent application No. 1994-9395

PTL 2; Japanese patent application No. 1997-110693

PTL 3: Japanese patent No. 3628647

PTL 4: Japanese patent application No. 1998-158161

PTL 5: Japanese patent application No. 2000-26288

PTL 6: PCT (WO) 2004-528317

PTL 7: Japanese patent application No. 2005-263695

PLT 8: Japanese patent application No. 2007-9166

PLT 9: Japanese patent application No. 2010-189440

PLT 10: Japanese patent No. 3120965

PLT 11: Japanese patent application No. 2001-302504.

PLT 12: U.S. Pat. No. 4107331

PLT 13: Japanese patent application No. 2008-273984

PTL 14: Japanese patent No. 4012328

### Non-patent Literature

NPL 1: Toshio Matsuda, Shokuhin Kigai Biseibutsu No Taagetto Seigyo [Targeted Control of Microorganisms Toxic to Food], pp. 31-41, Saiwai Shobo (Japan), 2009

NPL 2: Shigeki Yamamoto et al., Biseibutsu Sakkin Jitsuyou Deeta Shuu [Compilation of Data on Microbe Sterilization], pp. 362-363, Science Forum (Japan), 2005.

NPL 3: IPCS (WHO), Concise International Chemical Assessment Document No. 26, Benzoic Acid and Sodium Benzoate, p. 11, Division of Safety Information on Drug, Food and Chemicals of National Institute of Health Sciences (Japan), 2007

NPL 4: Foley, E. J. and Lee, S. W., J. Invest. Dermatol., 10, 249 (1948)

NPL 5: Shomatsu Osada, Toni Kojima, Kuensan de Ishairazu [Citric Acid Keeps the Doctor Away], p. 114, Nitto Shoin (Japan), 2005

NPL 6: Toshio Matsuda, Toshihiro Yano, Masahiro Maruyama, Hidehiko Kumagai, Yuuki Sanrui No Koukin Sayo—Kakushu pH Ni Okeru Saishou Hatsuiku Soshi Noudo No Kentou [Antimicrobial Action of Organic Acids—Study of Minimum Inhibitory Concentration at Various pH], Journal of Japanese Society for Food Science and Technology (Japan), 41, 687 (1994)

NPL 7: IPCS (WHO), Concise International Chemical Assessment Document No. 26, Benzoic Acid and Sodium Benzoate, p. 4, National Institute of Health Sciences (Japan), 2007

NPL 8: lyakuhin Tenkabutsu Handbook, (Pharmaceutical Additives Handbook) p. 100, Editosan, Koukinkassei [Edetate, Antimicrobial Activity], Koji Nagai supervising editor, Yakujinipposha (Japan), 2001

NPL 9: B. Chudzik, A. Malm, B. Rajtar and M. Polz-Dacewicz, Annals of Microbiology, 57 (1), 115-119 (2007)

NPL 10: L. Abrunhosa and A. Venancio, Asian J. Biochemistry, 3 (3), 176-181 (2008)

NPL 11 Kaoru Nakajima, Rintaro Terada, Minoru Kubota, Shihan EDTA Seizai no Koukin-Sei [Antibacterial Effect of Commercial EDTA Formulation], The Proceedings of 125th Japanese Society of Conservative Dentistry, p. 144 (2006)

### SUMMARY OF INVENTION

### Technical Problem

Dermatomycosis is a fungal infection that primarily affects the horny layer of the skin, and is typified by dermatophytosis (e.g., tinea pedis). The dermatophytosis that affects animals and people is an infection common to both, and it is known to be very difficult to cure even with medication. It is unknown why the dermatophytes that parasitize the shallow portions of the skin are difficult to kill; however, one of the factors is believed to be that synthetic antifungal agents that are used in the field of dermatology, such as azole antifungal agents and allylamine antifungal agents, do not penetrate well into the horny layer. Although it has been shown in in vitro drug-susceptibility tests that such antifungal agents have a strong antifungal action against dermatophytes, if the medication does not reach the portions where the dermatophytes are located, the tinea will not be cured. Accordingly, the development of a new type of medication with superior skin-penetrating ability has been desired. It is hoped that such a medication with the ability to permeate deep into the skin will act only to cure tinea. Additionally, although treatments for tinea unguium are being performed using orally administered antifungal agents, since such oral treatment causes side effects such as liver damage and the like, there is a need to develop a treatment that can treat and/or cure tinea unguium by way of a topically administered medication. The present invention provides an effective pharmaceutical composition for dermatomycoses such as, in general, dermatophytosis, cutaneous candidiasis, and tinea versicolor.

The object of the present invention is to provide a new pharmaceutical composition for treating dermatomycoses, especially dermatophytosis.

### Solution to Problem

It is clear that in order to solve the above-described problems, research into antifungal agents from a viewpoint completely different from that of the previously utilized method of researching analogs of synthetic antifungal agents is necessary. Conventional synthetic antifungal agents are generally insoluble or very poorly soluble in water, and it is believed that this is an important factor of why these agents lose their ability to permeate into the skin. In order to improve the permeability into the skin, it would be preferable to select a highly water-soluble compound. Acetic acid has high permeability into the skin and is an excellent antifungal agent. After investigating drug susceptibility of dermatophytes using various organic acids according to the guidelines of the Clinical Laboratory Standards Institute (CLSI), US, we determined that citric acid has an antimicrobial effect against dermatophytes. Since these drug-susceptibility tests were performed under neutral conditions using 3-morphorinopropanesuolfonic acid (MOPS) as a buffer, the citric acid in the test system was in the form of non-acidic citric acid trianions; it was shown that such citric acid trianions have an antimicrobial effect against dermatophytes.

Accordingly, when we investigated the antimicrobial effects of various salts of organic acids against various pathogenic fungi (e.g., dermatophytes, Candida, Malassezia, Aspergillus, and Cryptococcus), it was discovered that salts of benzoic acid, sorbic acid, and ethylenediaminetetraacetic acid had a strong antimicrobial action. From such results, it was determined for the first time that certain kinds of organic acid salts have an antimicrobial effect against a broad range of pathogenic fungi. As such organic acid salts, potassium salts, calcium salts, and magnesium salts that are highly soluble in water can be selected as appropriate. It was determined that these organic acid salts have high permeability into the skin, and are strongly effective in treating and/or curing tinea of the skin. It was also shown that these organic acid salts have a superior effect when applied topically against tinea unguium, and were demonstrated to easily treat and/or cure tinea unguium. Moreover, it was shown that these organic acid salts have superior therapeutic effects against skin candidiasis, Candida tinea unguium, and malasseziosis.

The pharmaceutical composition according to the present invention is characterized by comprising, as an active ingredient, sodium benzoate, potassium benzoate, magnesium benzoate, calcium benzoate, potassium sorbate, sodium sorbate, calcium sorbate, ethylenediaminetetraacetic acid tetrasodium salt, ethylenediaminetetraacetic acid tetrapotassium salt, ethylenediaminetetraacetic acid trisodium salt, ethylenediaminetetraacetic acid tripotassium salt, ethylenediaminetetraacetic acid disodium salt, ethylenediaminetetraacetic acid dipotassium salt, or ethylenediaminetetraacetic acid magnesium disodium salt. In a preferred embodiment, the pharmaceutical composition comprises at least one pharmaceutically acceptable salt of ethylenediaminetetraacetic acid. In a preferred embodiment, the pharmaceutically acceptable salt of ethylenediaminetetraacetic acid is at least one member selected from the group consisting of ethylenediaminetetraacetic acid tetrasodium salt, ethylenediaminetetraacetic acid tetrapotassium salt, ethylenediaminetetraacetic acid trisodium salt, ethylenediaminetetraacetic acid tripotassium salt, ethylenediaminetetraacetic acid disodium salt, ethylenediaminetetraacetic acid dipotassium salt, and ethylenediaminetetraacetic acid magnesium disodium salt.

Organic acids alone are acidic and strongly irritate the skin, which makes them difficult to use as a treatment for fungal conditions such as tinea. However, such acidity is lost or at least weakened in organic acid salts, which means that there is no irritation of the skin, and that such salts can be safely used as pharmaceutical compositions for tinea. The aforementioned salts have a high therapeutic effect against dermatophytosis and the like, and have few side effects.

In one embodiment, the pharmaceutical composition preferably comprises at least one member selected from the group consisting of sodium benzoate, potassium benzoate, potassium sorbate, sodium sorbate, ethylenediaminetetraacetic acid trisodium salt, ethylenediaminetetraacetic acid tripotassium salt, ethylenediaminetetraacetic acid tetrasodium salt, ethylenediaminetetraacetic acid tetrapotassium salt, ethylenediaminetetraacetic acid disodium salt, and ethylenediaminetetraacetic acid dipotassium salt.

In one embodiment, it is preferable for the organic acid salts mentioned above to be dissolved or dispersed in a liquid medium, and for the pharmaceutical composition to be used by being administered to an affected area by immersing the areas affected by dermatomycosis in the pharmaceutical composition, or by spraying the pharmaceutical composition onto an affected area.

### ADVANTAGEOUS EFFECTS OF INVENTION

The present invention is effective in treating and/or curing dermatomycosis, and/or in preventing recurrence of dermatomycosis.

### DESCRIPTION OF EMBODIMENTS

The pharmaceutical composition for dermatomycosis according to the present invention preferably penetrates the skin (e.g., horny layer) and/or nails, and has been determined to easily treat and/or cure dermatophytosis (e.g., tinea pedis), which had previously been difficult. By applying the pharmaceutical composition of the present invention to the areas of the skin affected by dermatophytes 1-3 times a day, the concentration of the drug within the skin that is required to kill the dermatophytes is maintained, which enables the dermatophytes to be eliminated; accordingly, the tinea can be treated and/or cured by continuing the process for several days to several months. Additionally, by using the pharmaceutical composition of the present invention, tinea unguium was successfully cured by topical application. In order to cure tinea unguium by way of topical application, an effective method is to immerse the entire nail for a sufficient period of time in the pharmaceutical composition of the present invention, which enables the dermatophytes present inside the nail to be effectively sterilized or eliminated. This method of immersing the nail involves performing the process once a day, preferably for 1-5 hours or at bedtime. If the process is consecutively performed 1-15 times, the dermatophytes inside the nail will be eliminated. Afterward, new nail will grow over time, enabling recovery with healthy nail. Because tinea unguium usually involves the presence of a dermatophyte infection in the skin surrounding the nail, this process of immersing the entire nail in the medicinal solution should preferably be continued for several months. Conventionally, tinea unguium could only be cured by intake of an antifungal agent through oral administration. With the pharmaceutical composition of the present invention, it is now possible to treat and/or cure tinea unguium by a topical application therapy. Additionally, although Candida can often be separated together with dermatophytes from nail samples taken for tinea unguium, if it is believed that a mixed infection of both tinea and Candida is occurring, the compounds according to the present invention exhibit sufficient therapeutic effectiveness against such tinea cases that involve a mixed infection. A preparation containing the compounds according to the present invention shows a pH close to neutral, and because irritation to the skin due to acidity derived from organic acid is absent or very weak, the compounds according to the present invention have superior characteristic in that inflammation of the skin does not readily occur. In this way, the pharmaceutical composition for dermatomycosis according to the present invention contributes positively to human society by being an ideal pharmaceutical composition for tinea. Moreover, the pharmaceutical composition for dermatomycosis according to the present invention is also an effective treatment for Malassezia infections, as it is clear that malasseziosis of the skin is easily cured by this pharmaceutical composition.

The drug concentration of the compounds according to this invention is selectable from any concentration within a range that kills pathogenic fungi. It is preferable to facilitate permeation of anions of organic acid into the skin and to maintain the concentration for a long time. In the case of tinea unguium, it is preferable to make the anions of organic acid sink deeply into the nail so as to reach the parts inhabited by the dermatophytes. From this point of view, it is preferable that the concentration of the pharmaceutical composition for dermatomycosis is 0.001-25 wt %, more preferably 0.01-20 wt %, and still more preferably 0.1 to 10 wt %. If the drug concentration is too low, the treatment will not be sufficiently effective against the pathogenic fungi, resulting in the need to increase the frequency of the application of the pharmaceutical composition. Conversely, if the concentration is too high, there is increased irritation of the skin, leading to potential inflammation on sensitive parts of the skin, which can impair the therapeutic effect against the dermatomycosis. In addition, the daily intake tolerance of benzoic acid salts and sorbic acid salts as food preservatives are established by the World Health Organization. In the case of benzoic acid salts, the daily intake tolerance is set at 5 mg or less (benzoic acid content) per 1 kg in weight, and in the case of sorbic acid salts, it is set at 25 mg or less (sorbic acid content) per 1 kg weight. Furthermore, in an evaluation of the safety of food additives conducted by the Joint FAO/WHO Expert Committee on Food Additives (JECFA), the permissible daily intake of edetate disodium was set at not greater than 2.5 mg per 1 kg of body weight; these standard must be complied with.

The salt of organic acids of the present invention have the degree of water solubility required for the pharmaceutical composition. Accordingly, the appropriate type of formulation can be selected from a variety of forms such as a solution, a cream, an ointment, a spray, an emulsion, a gel, a foam, a paste, and an aerosol. The solvent for dissolving the compounds of the present invention can be any solvent in which the compounds are soluble, such as water, ethanol, isopropanol, glycerin, ethylene glycol, propylene glycol, macrogol, etc. The pharmaceutical composition for dermatomycosis according to the present invention can be used as a mixture with an existing synthetic antifungal agent, and there is nothing to prevent this pharmaceutical composition from being mixed in the desired proportions with a traditional tinea remedy such as pyroligneous acid, bamboo vinegar, acetic acid, or a plant extract liquid. Additionally, the pharmaceutical composition can suitably include additives generally used in topical formulations.

Excipients that can be used to formulate the pharmaceutical composition include gel-forming substances (e.g., gelatin, tragacanth, cellulose derivatives, alginates, and polyacrylic acid); moistening agents (e.g., urea, glycerol, and propylene glycol); contact-adhesive polymers (e.g., polyacrylates, and adhesive resins); ointment bases (e.g., petroleum jelly, fats, cellulose derivatives, polyacrylic acid, and polyethylene glycols); emulsifiers (e.g., wool wax, sorbitan esters, and monoglycerides); preservatives (e.g., benzalkonium chloride); antioxidants (e.g., butyl hydroxyanisole); thickeners (e.g., hydroxypropylmethylcellulose); pH correctants; binders (e.g., polyvinylpyrrolidone, starch, hydroxypropylmethylcellulose, and polyethylene glycols); fillers (e.g., microcrystalline cellulose and sorbitol); dyes; aroma substances; sweeteners (e.g., sorbitol and aspartame); solvents (e.g., water, ethanol, and ethanol-water mixtures); solubilizers (e.g., glycerol and propylene glycol); skin-penetration improvers (e.g., propylene glycol); plasticizers (e.g., sorbitol, glycerol, and phthalates); wetting agents (e.g., sodium lauryl sulphate, polysorbate); synthetic and natural oils (e.g., medium-chain triglycerides); and propellants for aerosol or foam sprays (e.g., norflurane, cryofluorane, dichlorofluoromethane, trichlorofluoromethane, propane, butane, isobutane, and nitrogen).

The antimicrobial activity of the pharmaceutical composition for dermatomycosis according to the present invention was evaluated through drug-susceptibility tests against major pathogenic fungi performed in compliance with the guidelines of the Clinical Laboratory Standards Institute (CLSI), USA. The pathogenic fungi selected were genus Tricophyton (T. rubrum, T. mentagrophytes, T. tonsurans), genus Microsporum (M. gypseum), genus Epidermophyton (E. floccosum), genus Candida (C. albicans), genus Aspergillus (A. fumigatus), and genus Cryptococcus (C. neoformans). The examination results are shown in Examples 1-4, and the primary results for the antimicrobial activity (MIC, mg/ml) of the compounds according to the present invention against T. mentagrophytes, which is a representative dermatophyte, were favorable, as follows: sodium benzoate, 0.20; potassium sorbate, 0.39; ethylenediaminetetraacetic acid trisodium salt, 0.078; ethylenediaminetetraacetic acid tetrasodium salt, 0.039. Additionally, drug-susceptibility testing was conducted against a member species (M. furfur) of the genus Malassezia performed in compliance with the methodology of Sugita et al. using the agar dilution method (T. Sugita et. al., J. Clinical Microbiology, 43, 2824-2829 (2005). The antimicrobial activity of the major compound of the present invention against M. furfur (MIC, mg/ml) produced favorable results as follows: sodium benzoate, 0.31; potassium sorbate, 0.31; ethylenediaminetetraacetic acid trisodium salt, 0.063 (Example 5). Since the compounds of the present invention are water-soluble and deeply penetrate the skin, these MIC values can serve as an index for setting the clinical doses. In the drug-susceptibility tests, Mg ions and Ca ions necessary for conidia (spores) of the fungus to germinate growth were included in the nutrient medium. Since the carboxyl group of the organic acids binds with these ions, the organic acids have a competitive inhibition action against the growth of fungi. A characteristic of the compounds of the present invention is that they have a chemical structure that causes them to bind strongly to such ions, and it is believed that by eliminating these ions from the test system, the compound impedes the growth of fungi and thereby exhibits an antimicrobial action. The fact that organic acid salts that are capable of binding strongly with magnesium ions and calcium ions exert an antimicrobial action against fungi overturns the conventional wisdom that non-disassociated organic acids exert antimicrobial activity. Since the compounds of the present invention exhibit an antimicrobial action by binding primarily with Mg ions and Ca ions, the compounds can exert an effective antimicrobial effect in the stratum corneum, where such ions are not easily supplied from within the blood. On the other hand, it is also apparent that as these ions exist in abundance in the internal organs and the like, the compounds lose their effectiveness, and it is believed that the compounds of the present invention will be ineffective against deep mycosis. Additionally, there is a theory that the antimicrobial effect of edetate salts against Candida is due to the edetate salts binding with zinc ions, thereby depleting the zinc (Patent Literature 12); this is considered to be correct in the case described here.

The compounds of the present invention were investigated with respect to the treatment of dermatomycosis affecting humans. The compounds of the present invention exhibited favorable curative effects against dermatophytosis (e.g., tinea), a typical dermatomycosis. The cases of tinea used in the investigation involved selection of various types of tinea such as vesicular-type tinea, interdigital-type tinea, hyperkeratotic tinea, tinea corporis, tinea unguium, etc. As shown in the examples, the pharmaceutical composition including the compounds of the present invention exhibits superior tinea-treating effects, and a complete cure of tinea can also be achieved. Curing of dermatophytosis using a pharmaceutical composition containing organic acid salts has been previously completely unknown, and has become clear for the first time due to the present invention.

In one embodiment, dermatomycosis treated by the pharmaceutical composition is preferably dermatophytosis. Dermatophytosis is preferably caused by at least one dermatophyte selected from the group consisting of those belonging to the genus Microsporum, the genus Trichophyton, or the genus Epidermophyton. Dermatophytes of the genus Microsporum include Microsporum audouinii, Microsporum canis, Microsporum equinum, Microsporum nanum, and Microsporum versicolor. Dermatophytes of the genus Trichophyton include Trichophyton rubrum, Trichophyton mentagrophytes, Trichophyton verrucosum, Trichophyton tonsurans, Trichophyton equinum, Trichophyton kanei, Trichophyton raubitschekii, and Trichophyton violaceum. Dermatophytes of the genus Epidermophyton include Epidermophyton floccosum. In a preferred embodiment, dermatophytosis is caused by at least one dermatophyte selected from the group consisting of Trichophyton rubrum, Trichophyton mentagrophytes, Trichophyton verrucosum, and Trichophyton tonsurans.

In a preferred embodiment, dermatophytosis that is treated according to the invention is tinea. There are various types of tinea, including tinea capitis, tinea corporis, tinea cruris, tinea manus, hyperkeratotic tinea, tinea pedis, and tinea unguium. In one embodiment, tinea is preferably hyperkeratotic tinea and/or tinea unguium.

The benzoic acid salt, the sorbic acid salt, the ethylenediaminetetraacetic tetrasubstituted salt, the ethylenediaminetetraacetic trisubstituted salt or the ethylenediaminetetraacetic disubstituted salt disassociate in the presence of water into benzoic acid anion, sorbic acid anion, ethylenediaminetetraacetic acid tetraanions, ethylenediaminetetraacetic acid trianions, or ethylenediaminetetraacetic acid dianions, respectively. These organic acid anions easily penetrate into the skin and eliminate dermatophytes. All of these organic acid anions exhibit a favorable therapeutic effect against dermatophytosis by depriving dermatophytes of metallic ions (e.g., magnesium ions, calcium ions, and the like), thereby destroying the fungus. It is believed that the ethylenediaminetetraacetic acid anions are able to migrate to the stratum corneum due to the destruction of the horny layer and the horny layer barrier by fungi in the affected area where dermatomycosis is observed. The ethylenediaminetetraacetic acid anions in the stratum corneum bind with essential metal elements such as Mg, Ca, Zn, and Fe, depleting them and thereby killing the fungus parasitizing the stratum corneum. Accordingly, ethylenediaminetetraacetic acid salts create an environment in the stratum corneum in which fungi are unable to survive. For treating dermatophytosis, depriving magnesium ion and calcium ion is more important than depriving zinc ion. Interestingly, depriving zinc ion is not considered to be effective in treating dermatophytosis, unlike in the treatment of candidiasis.

In one embodiment, a pharmaceutical composition comprising at least one pharmaceutically acceptable salt of ethylenediaminetetraacetic acid is prepared by dissolving at least one pharmaceutically acceptable salt of ethylenediaminetetraacetic acid in a suitable solvent. Accordingly, in one embodiment, the at least one pharmaceutically acceptable salt of ethylenediaminetetraacetic acid is present in the pharmaceutical composition as a dissociated anion.

The dermatophytes that cause dermatophytosis, a typical form of dermatomycosis, are mainly Trichophyton rubrum and Trichophyton mentagrophytes. For each kind of tinea in the examples of the present invention, it was confirmed that dermatophytes were the cause of the mycosis. This confirmation of dermatophytes involved planting a sample of skin or nail of an area affected with tinea in a Candida nutrient medium (Nissui Pharmaceutical Co., Ltd.), isolating the dermatophytes that grow, and then confirming the dermatophytes through gene analysis or observation of morphology. A more detailed description of the fungus identification is provided in the examples.

Dermatomycosis includes dermatophytosis, cutaneous candidiasis, and malasseziosis. As time progresses with these diseases, a variety of fungi will parasitize the site. Separation and genetic analysis of fungi were performed on red papules of tinea glabrosa, and the following pathogenic fungi were detected: Aspergillus (Aspergillus japonicas, Aspergillus tubingensis); Basidiomycete (Coprinellus velatopruinatus, Irpex lacteus, Schizophyllum commune, Ceriporia lacerata, Fomitopsis sp.); and others (Nigrospora sp., Paecilomyces variotii, Pleosporales family, Rhodotorula mucilaginosa). This shows that even in cases of tinea unguium or hyperkeratotic tinea, there is a possibility for these miscellaneous fungi to become pathogenic. These red papules were easily cured with pigmentation within 1-2 weeks through treatment with 2% solution of ethylenediaminetetraacetic acid trisodium salt (Example 23). This fact clearly indicates that ethylenediaminetetraacetic acid salts are effective in treating a variety of dermatomycoses.

Treatment of dermatomycosis, preferably dermatophytosis, is preferably performed by topically administering to the affected area of a patient suffering from dermatomycosis a pharmaceutical composition comprising at least one pharmaceutically acceptable salt of ethylenediaminetetraacetic acid. The patient can be any animal that develops dermatomycosis, and is preferably human. In one embodiment, the treatment of dermatomycosis can be carried out by administering the pharmaceutical composition at a frequency of at least once daily, or preferably at least twice daily. The at least once-daily administration can be continued until the desired level of treatment is achieved. For example, the at least once-daily administration can be continued for at least one week, two weeks, three weeks, four weeks, one month, two months, three months, four months, five months, six months, one year, or two years. The desired level of treatment can be a complete cure of dermatomycosis (preferably dermatophytosis), which preferably prevents recurrence of any symptoms associated with dermatomycosis.

The term “treatment” mean at least the mitigation of a disease condition or symptom associated with dermatomycosis (preferably, dermatophytosis) in a subject. The term “treatment” includes curing, healing, inhibiting, relieving, improving, and/or alleviating, in whole or in part, the disease condition. The mitigation of a disease condition or symptom may be about 100%, 99%, 98%, 97%, 96%, 95%, 90%, 80%, 70%, 60%, 50%, 40%, 30%, 20%, 10%, 5%, or 1% in the subject, versus a subject to which the pharmaceutical composition disclosed herein has not been administered. In one embodiment, treatment means reducing the population of dermatophytes causing the disease in the subject to an undetectable level, where detection is by any conventional means, such as culturing a sample in the laboratory. In another embodiment, treatment means complete cure of the infection, shown by an absence of clinical symptoms associated with dermatomycosis.

Based on the above description, the present invention includes the following aspects.

(1) A dermatomycosis therapeutic agent characterized by being a topical agent containing, as an active ingredient, at least one organic acid salt selected from the group consisting of benzoic acid salts, sorbic acid salts, and ethylenediaminetetraacetic acid salts.
(2) The dermatomycosis therapeutic agent according to (1), in which the organic acid salt is an alkali metal salt or an alkaline earth metal salt of an organic acid.
(3) The dermatomycosis therapeutic agent according to (1) or (2), in which the benzoic acid salt is selected from sodium benzoate, potassium benzoate, magnesium benzoate, and calcium benzoate.
(4) The dermatomycosis therapeutic agent according to (1) or (2), in which the sorbic acid salt is selected from sodium sorbate, potassium sorbate, and calcium sorbate.
(5) The dermatomycosis therapeutic agent according to (1) or (2), in which the ethylenediaminetetraacetic acid salt is selected from ethylenediaminetetraacetic acid tetrasodium salt, ethylenediaminetetraacetic acid tetrapotassium salt, ethylenediaminetetraacetic acid trisodium salt, ethylenediaminetetraacetic acid tripotassium salt, ethylenediaminetetraacetic acid disodium salt, ethylenediaminetetraacetate dipotassium salt, and ethylenediaminetetraacetate magnesium disodium salt.
(6) The dermatomycosis therapeutic agent according to any of (1) to (5), in which the topical agent is a liquid.
(7) The dermatomycosis therapeutic agent according to (6), in which the organic acid salt is dissolved or dispersed in a liquid medium, and the dermatomycosis therapeutic agent is administered to an affected area of dermatophytosis by immersing the affected area of a subject in a therapeutic agent for dermatophytosis.
(8) The dermatomycosis therapeutic agent according to any of (1) to (7), in which the dermatomycosis is dermatophytosis.
(9) The dermatomycosis therapeutic agent according to any of (1) to (7), in which the dermatomycosis is dermatophytosis with complications of candidiasis.
(10) The dermatomycosis therapeutic agent according to any of (1) to (7), in which the dermatomycosis is malasseziosis.
Item 1. A method for treating dermatophytosis in a subject, comprising topically administering to the subject a therapeutically effective amount of a pharmaceutical composition comprising at least one pharmaceutically acceptable salt of ethylenediaminetetraacetic acid.
Item 2. The method of Item 1, wherein the at least one pharmaceutically acceptable salt of ethylenediaminetetraacetic acid is selected from the group consisting of ethylenediaminetetraacetic acid tetrapotassium salt, ethylenediaminetetraacetic acid trisodium salt, ethylenediaminetetraacetic acid tripotassium salt, ethylenediaminetetraacetic acid disodium salt, ethylenediaminetetraacetic acid dipotassium salt, and ethylenediaminetetraacetic acid magnesium disodium salt.
Item 3. The method of Item 1 or 2, wherein the dermatophytosis is caused by at least one dermatophyte selected from the group consisting of dermatophytes belonging to the genus Microsporum, the genus Trichophyton, or the genus Epidermophyton.
Item 4. The method of any one of Items 1 to 3, wherein the dermatophytosis is tinea.
Item 5. The method of Item 4, wherein the tinea is at least one tinea selected from the group consisting of tinea capitis, tinea corporis, tinea cruris, tinea manus, tinea pedis, hyperkeratotic tinea, and tinea unguium.
Item 6. The method of claim 4, wherein the tinea is tinea unguium.
Item 7. The method of claim 4, wherein the tinea is hyperkeratotic tinea.
Item 8. The method of any one of Items 1 to 7, wherein the pharmaceutical composition is in the form of a solution, cream, gel, ointment, or emulsion.
Item 9. The method of any one of Items 1 to 8, wherein the pharmaceutical composition is in the form of a solution.
Item 10. The method of Item 9, wherein the topically administering comprises immersing an infected area of dermatophytosis of the patient in the solution.
Item 11. The method any one of Items 1 to 9, wherein the topically administering comprises spraying the solution to an infected area of dermatophytosis of the patient.
Item 12. The method of any one of Items 1 to 11, wherein the pharmaceutical composition comprises the at least one salt of ethylenediaminetetraacetic acid at a concentration of 0.001 to 25 wt %.
Item 13. The method of any one of Items 1 to 12, wherein the subject is a human subject.
Item 14. The method of any one of Items 1 to 13, wherein the pharmaceutical composition is administered to the patient at least once daily.
Item 15. The method of any one of Items 1 to 14, wherein the pharmaceutical composition is administered to the patient at least once daily for at least one week.
Item 16. The method of any one of Items 1 to 15, wherein the method cures the dermatophytosis.
Item 17. The method of any one of Items 1 to 16, wherein the method prevents recurrence of the dermatophytosis.

### EXAMPLES

Although the antifungal activity of the compounds according to the present invention, as well as the therapeutic action against tinea and against candidiasis will now be described by way of examples, the effects of the invention are not limited to such examples. In the following examples, the percentages are weight percentages.

### Example 1

The antifungal activity of the compounds of the present invention was evaluated by performing drug-sensitivity tests against various pathogenic fungi. The testing method was a broth microdilution method in conformance with the guidelines of the Clinical Laboratory Standards Institute (CLSI), USA. The antifungal activity of sodium benzoate against various types of pathogenic fungi (fungal name, fungal number, and MIC (mg/ml) shown together) was as follows: T. rubrum IFM 59814, 0.39; T. mentagrophytes IFM 59813, 0.20; M. gypseum IFM 59816, 1.6; E. floccosum IFM 53345, 0.20; C. albicans IFM 5740, 6.3; A. fumigatus IFM4942, 3.1; C. neoformans IFM5807, 1.6. The antifungal activity (MIC (mg/ml)) of other benzoic acid salts against T. mentagrophytes IFM 59813 was as follows: potassium benzoate, 0.78; magnesium benzoate, 0.78. In addition, in the test using calcium benzoate, a deposition thought to be calcium benzoate occurred within the test system, and the antifungal activity thereof could not be measured; however, a 1% aqueous solution of calcium benzoate showed a curative effect against tinea of the skin (Example 15).

In addition, because the compounds of the present invention have a new antimicrobial mechanism, there is no control medicine that can be used to contrast and compare these test results.

### Example 2

The antifungal activity of sorbic acid salts was evaluated in the same manner described in Example 1. The antifungal activity of potassium sorbate against various types of pathogenic fungi (fungal name, fungal number and MIC (mg/ml) shown together) was as follows: T. rubrum IFM 59814, 0.78; T. mentagrophytes IFM 59813, 0.39; M. gypseum IFM 59816, 0.78; E. floccosum IFM 53345, 0.20; C. albicans IFM 5740, 3.1; A. fumigatus IFM4942, 0.78; C. neoformans IFM5807, 0.20. The antifungal activity (MIC (mg/ml)) of sodium sorbate against T. mentagrophytes IFM 59813 was 0.78. In addition, in the test using calcium sorbate, deposition thought to be calcium benzoate occurred within the test system, and the antifungal activity thereof could not be measured; however, a 1% aqueous solution of calcium sorbate showed a curative effect against tinea of the skin (Example 15).

### Example 3

The antifungal activity of ethylenediaminetetraacetic acid salts was evaluated in the same manner described in Example 1. The antifungal activity of ethylenediaminetetraacetic acid trisodium salt against various types of pathogenic fungi (fungal name, fungal number and MIC (mg/ml) shown together) was as follows: T. rubrum IFM 59814, 0.039; T. mentagrophytes IFM 59813, 0.078; M. gypseum IFM 59816, 0.078; E. floccosum IFM 53345, 0.078; C. albicans IFM 5740, 0.00020; A. fumigatus IFM4942, 0.0098; C. neoformans IFM 5807, 0.0098. In addition, the antimicrobial activity (MIC (mg/ml)) of ethylenediaminetetraacetic acid tripotassium salt against T. mentagrophytes IFM 59813 was 0.078.

The results of testing the antifungal activity (MIC (mg/ml)) of ethylenediaminetetraacetic acid tetrasodium salt, ethylenediaminetetraacetic acid disodium salt and ethylenediaminetetraacetic acid magnesium disodium salt against T. mentagrophytes IFM 59813 were respectively 0.039, 0.078, and 0.078.

### Example 4

The antifungal activity of the compounds of the present invention against Malassezia spp. was evaluated in drug sensitivity tests against human-related genus Malassezia species, performed in compliance with the methodology of Sugita et al. (2005). The test drug and mLNA nutrient medium were added to the wells of a 24-well microtiter plate with a total quantity of 2 mL. A 50 μL microbe suspension with a concentration of approximately 1×104 cell/mL was added to the medium, which was cultured aerobically for 7 days at 32° C. In comparison to the control (no test drug), the density required to completely prevent growth was set as the minimum inhibitory concentration (MIC). The antifungal activity (MIC, mg/ml) of the compound of the present invention against M. furfur was as follows: sodium benzoate, 0.31; potassium sorbate, 0.31; ethylenediaminetetraacetic acid trisodium salt, 0.063. The antifungal activity against various Malassezia species of ethylenediaminetetraacetic acid trisodium salt (fungus and MIC, (mg/ml) shown together) was: M. globosa, 0.063; M. restricta, 0.031; M. sympodialis, 0.063; M. dermatitis, 0.031; M. obtusa, 0.031; M. slooffiae, 0.063; M. japonica, 0.063; and M. yamatoensis, 0.063.

Example 5

To 46.0 g of ethylenediaminetetraacetic acid trisodium salt trihydrate was added 20 ml of glycerin (84-87% aqueous solution), and 15% ethanol was further added to prepare 2 L of a 2% test solution. This test solution was applied twice daily by spraying onto parts of the arch on the sole of the foot and side portions thereof where hyperkeratotic type tinea was widespread. Due to this process, lesions, which at that point had not come to the surface, came to the surface. After continuing the process for approximately 2 months, hyperkeratotic-type tinea, which continually reoccurred and had been difficult to cure, was eliminated, and clean skin was recovered. Dermatophyte and Candida were isolated from the parts affected with tinea in this case, and the dermatophyte was confirmed from spiral-shaped hyphae and the form of the conidia as being Trichophyton mentagrophytes.

Example 6

To 57.5 g of ethylenediaminetetraacetic acid trisodium salt trihydrate was added 50 ml of glycerin (84-87% aqueous solution), and then 15% ethanol was further added to prepare 5 L of a 1% test solution. A foot widely affected by hyperkeratotic-type tinea on the sole, the sides, the toes, between the toes, and on the instep parts of the foot was immersed in this 1% preparation for 1 hour a day. As this process proceeded, the hyperkeratotic skin gradually peeled off, and pink skin was revealed. The tinea that had covered the entire foot was eliminated, and healthy skin returned about one and a half months later.

A 2% test solution of sodium benzoate was prepared in a similar procedure, and the other foot having similar symptoms was immersed in this 2% preparation for 1 hour a day. The tinea was eliminated, and healthy skin returned about two months later.

### Example 7

A 2% test solution of ethylenediaminetetraacetic acid tetrasodium salt, prepared in the same manner as Example 5, was applied by spraying once daily onto an area between the ring toe and the little toe affected by interdigital tinea. After approximately 2 months, the symptoms on the skin had disappeared, and healthy skin returned. Dermatophyte was isolated from the affected area of the skin, and gene analysis was performed. It was determined that the base sequence of the rDNA ITS domain indicated Trichophyton rubrum.

Example 8

A 1% test solution of ethylenediaminetetraacetic acid trisodium salt prepared in the same manner as Example 6 was applied by spraying once a day onto an area, approximately 5 cm wide and 3 cm long, of a chest affected by tinea corporis, which was denoted by flaring redness. After approximately 2 weeks of this process, the areas affected with red flaring had disappeared, and healthy skin returned. As dermatophyte was isolated from an affected area of swollen redness that was the source of the lesion, gene analysis was performed, and it was determined that the base sequence of the rDNA ITS domain indicated Trichophyton mentagrophytes.

Example 9

A 1% test solution of ethylenediaminetetraacetic acid trisodium salt prepared in the same manner as Example 6 was applied by spraying once a day to an area, approximately 10 cm wide and 5 cm long, of the left thigh where the subject had tinea corporis, denoted by a cluster of red spots several mm in diameter. After approximately one month, symptoms on the skin had disappeared or cicatrized; accordingly, the tinea corporis was healed. Based on the characteristics of the symptoms in the skin, this tinea was considered to be caused by Microsporum canis (M. canis).

Example 10

To 119 mg of ethylenediaminetetraacetic acid tetrasodium salt tetrahydrate was added 20% ethanol to prepare 1 L of 0.01% test solution. This test solution was applied by spraying, three times a day, to tinea corporis denoted by red flaring ridges that had occurred on the thigh. After performing this process for approximately 2 weeks, the redness had disappeared, and changed to a pigment deposit. The tinea was cured.

Four 0.01% test solutions of sodium benzoate, potassium sorbate, ethylenediaminetetraacetic acid trisodium salt, and ethylenediaminetetraacetic acid disodium salt were prepared according to the same procedure, and were all confirmed to heal tinea corporis.

Example 11

To 230 g of ethylenediaminetetraacetic acid tetrasodium salt hydrate was added 100 ml of glycerin (84-87% aqueous solution), and then 15% ethanol was added to prepare 10 L of a 2% test solution. An appropriately sized fingerstall was made around the big toe, which had serious tinea unguium with discoloration and deformation affecting the entire nail, of the right foot. The 2% test solution was introduced into this stall, and the process was repeated to achieve continuous immersion for 3 days while supplementing the test solution as required, resulting in the release of a large quantity of small white flakes of tissue. After this process was continued for approximately 2 months, healthy nail grew from the nail base; after 6 months, recovery was complete with a healthy nail. Since dermatophyte was isolated from the affected areas of the nail, gene analysis was performed, and it was determined that the base sequence of the rDNA ITS domain indicated Trichophyton rubrum.

Example 12

In the same manner as Example 5, 2% test solution of ethylenediaminetetraacetic acid trisodium salt was prepared. Approximately 2 ml of this test solution was introduced into a commercially sold fingerstall (size M) into which was inserted about one-third of the end tip of the nail and the skin portion in front of the nail of a right-hand little finger affected by tinea. This soaking process was performed at bedtime. After repeating this process for 2 months, the skin of the nail floor portion peeled off several times before healthy skin and nail were recovered. In this case, dermatophyte and Candida fungus were isolated from the affected parts of the nail, and the dermatophyte was confirmed by morphological observation of the hyphae as being Trichophyton mentagrophytes.

Example 13

To 10 g of sodium benzoate was added 20% ethanol to prepare 1 L of 1% test solution. Approximately 2-3 ml of this test solution was introduced into a commercially sold fingerstall into which was inserted the entire nail of right-hand thumb affected by tinea unguium. This soaking process was performed for one day, and this treatment was performed 10 times. The test solution penetrated into the nail, and the entire nail swelled up and softened. The skin of the fingertip came off, indicating that the tinea of the skin was eliminated. New nail grew from the root of the nail, and restored healthy nail about 3 months later.

In addition, a test solution containing 1% of potassium sorbate, 1% of glycerin (84-87% aqueous solution), and 15% ethanol was prepared, and a right-foot, big-toe toenail entirely discolored white was immersed in this test solution for 1 hour a day. Healthy nail was regained about 9 months later.

Example 14

The 1% ethylenediaminetetraacetic acid trisodium salt test solution used in Example 10 was applied twice a day onto a light-brown area about 4×10 cm in size on the lower-left abdomen. After approximately 3 weeks, the light-brown area had disappeared. The light-brown section in this case was characterized by no itching or redness, and it was very similar to a photo of malasseziosis appearing in the Hifubyo Kara Atorasu [Skin Disease Color Atlas]. Therefore, it was considered to be a Malassezia infection.

Example 15

To 2.4 g of calcium benzoate trihydrate was added water to prepare 200 g of a 1% test solution. This solution was atomized twice a day for tinea glabrosa denoted by redness on the abdominal region. The skin symptoms disappeared about two weeks later, changing into a pigment deposit. The tinea was eliminated. Similarly, it was confirmed that a 1% solution of calcium sorbate allowed for complete recovery from tinea glabrosa.

Example 16

A 2% test solution of ethylenediaminetetraacetic acid trisodium salt prepared in the same manner as Example 5 was applied by spraying twice a day onto a left-foot, big-toe toenail affected by tinea unguium, against which oral therapy using Itrizole had been ineffective. As a result, new nail growth extended from the base of the nail. The portion of the nail affected by tinea was gradually discharged toward the tip, where it was cut off, thereby curing the tinea.

Example 17

A 2% test solution of ethylenediaminetetraacetic acid trisodium salt prepared in the same manner as Example 5 was applied by spraying twice a day onto hyperkeratotic tinea that extended from the base of the toes to the arch. As a result, the hyperkeratotic skin was gradually replaced by healthy skin. After approximately 6 months, the infected area had been entirely replaced by healthy skin. Dermatophyte was isolated from the skin in this case, which was confirmed to be Trichophyton rubrum through gene analysis.

Example 18

A 2% ethylenediaminetetraacetic acid trisodium salt test solution prepared in the same manner as in Example 5 was applied by spraying twice a day over small red papules occurring on the scalp at the back of the head. After 2 weeks, the papules had disappeared, and the area was smooth and completely healed.

Example 19

A 2% test solution of ethylenediaminetetraacetic acid trisodium salt prepared in the same manner as Example 5 was applied by spraying twice a day over small red papules occurring on the back of the right thigh. After 10 days, the papules had disappeared, changing to a pigment deposit; the condition had completely healed. Dermatophyte was isolated from the papules in this case, and was confirmed through gene analysis to be Trichophyton mentagrophytes.

Example 20

To 69.0 g of ethylenediaminetetraacetic acid trisodium salt trihydrate was added 20 ml of glycerin (84-87% aqueous solution), and a 15% ethanol aqueous solution was added to a total of 2 L. The crystals were dissolved to prepare a 3% test solution. This 3% liquid was applied by spraying twice a day onto a left-foot, little-toe toenail that was completely discolored. As a result, new nail grew out from the nail base, and the portion affected by tinea was gradually discharged toward the end. After approximately 8 months, the portion affected by tinea was discharged to the tip, where it was cut off; thereby, the tinea unguium was cured. Dermatophyte was isolated from the nail in this case, and, through gene analysis, was confirmed to be Trichophyton rubrum.

Example 21

To 115 g of ethylenediaminetetraacetic acid trisodium salt trihydrate was added 20 ml of glycerin (84-87% aqueous solution), and a 15% ethanol aqueous solution was added to a total of 2 L. The crystals were dissolved to prepare a 5% test solution. This 5% liquid was applied by spraying twice a day onto left and right heels that were affected by hyperkeratotic tinea. The skin that had become discolored white gradually disappeared, and after 3 months, there was complete recovery of healthy skin. The dermatophyte that was isolated from the skin in this case was confirmed through gene analysis to be Trichophyton rubrum.

Example 22

A mixture of 115 g of ethylenediaminetetraacetic acid trisodium trihydrate in 885 g of water was stirred to prepare a 10% test solution. This 10% test solution was applied once a day onto tinea unguium occurring in the end portion of the thumb of the right hand. The eroded portion of the nail was gradually pressed out towards the tip, and after about one month, healthy nail was recovered. The dermatophyte that was isolated from the nail in this case was confirmed through gene analysis to be Trichophyton rubrum.

Example 23

The pathogen in the small red papule of tinea glabrosa on the back of the head was separated, and it was confirmed by gene analysis to be Aspergillus japonicus. This red papule was treated twice a day with a 2% solution of ethylenediaminetetraacetic acid trisodium salt prepared in the same manner as in Example 5. The papule disappeared with pigmentation, and a complete cure was achieved two weeks later. Similarly, the pathogens in red papules of tinea glabrosa on the thigh, abdomen, breast, or back were confirmed to be Aspergillus tubingensis, Basidiomycete (Coprinellus velatopruinatus, Irpex lacteus, Schizophyllum commune, Ceriporia lacerata, Fomitopsis sp.), and others (Nigrospora sp., Paecilomyces variotii, Pleosporales family, and Rhodotorula mucilaginosa). These red papules were also cured completely using a 2% solution of ethylenediaminetetraacetic acid trisodium salt once or twice a day for 1-2 weeks.

A 20% test solution containing 230 g of ethylenediaminetetraacetic acid trisodium salt trihydrate, 10 ml of glycerin (84-87% aqueous solution), and 760 ml of water were prepared. The test solution was applied twice a day to a right-thumb thumbnail that was completely affected by tinea unguium. As the treatment progressed, new nail extended from the root of the nail, and healthy nail was restored about 1 year later. Trichophyton rubrum was detected in the nail of the affected part.

### INDUSTRIAL APPLICABILITY

The present invention relates to a pharmaceutical composition for dermatomycosis, and contains an effective pharmaceutical composition against dermatophytosis (tinea pedis), which is said to affect many people in Japan. Tinea has previously been very difficult to cure; however, by using the pharmaceutical composition for dermatomycosis according to the present invention, tinea can easily be completely cured. Additionally, the present invention creates a new method to completely cure tinea unguium, for which oral medication was thought to be the only cure, using a topical treatment. The present invention can relieve humanity from the suffering of dermatomycosis, and therefore has huge possibilities for industrial application.

1. A method for treating dermatophytosis in a subject, comprising topically administering to the subject a therapeutically effective amount of a pharmaceutical composition comprising at least one pharmaceutically acceptable salt of ethylenediaminetetraacetic acid.

2. The method of claim 1, wherein the at least one pharmaceutically acceptable salt of ethylenediaminetetraacetic acid is selected from the group consisting of ethylenediaminetetraacetic acid tetrasodium salt, ethylenediaminetetraacetic acid tetrapotassium salt, ethylenediaminetetraacetic acid trisodium salt, ethylenediaminetetraacetic acid tripotassium salt, ethylenediaminetetraacetic acid disodium salt, ethylenediaminetetraacetic acid dipotassium salt, and ethylenediaminetetraacetic acid magnesium disodium salt.

3. The method of claim 1, wherein the dermatophytosis is tinea unguium.

4. The method of claim 1, wherein the dermatophytosis is hyperkeratotic tinea.

5. The method of claim 1, wherein the pharmaceutical composition is in the form of a solution, cream, gel, ointment, or emulsion.

6. The method of claim 1, wherein the pharmaceutical composition is in the form of a solution.

7. The method of claim 6, wherein the topically administering comprises immersing an infected area of dermatophytosis of the patient in the solution.

8. The method of claim 6, wherein the topically administering comprises spraying the solution to an infected area of dermatophytosis of the patient.

9. The method of claim 1, wherein the pharmaceutical composition comprises the at least one salt of ethylenediaminetetraacetic acid at a concentration of 0.001 to 10%.

10. The method of claim 1, wherein the subject is a human subject.

11. The method of claim 1, wherein the pharmaceutical composition is administered to the patient at least once daily.

12. The method of claim 1, wherein the pharmaceutical composition is administered to the patient at least once daily for at least one week.

13. The method of claim 12, wherein the method cures the dermatophytosis.

14. The method of claim 12, wherein the method prevents recurrence of the dermatophytosis.
